(19) United States
(12) Patent Application Publication
(10) Pub. No.: US 2014/0242174 A1
(43) Pub. Date: Aug. 28, 2014

(54) TREATING COUGH AND TUSSIVE ATTACKS

(71) Applicant: Michael J.A. Walker, Vancouver (CA)

(72) Inventor: Michael J.A. Walker, Vancouver (CA)

(73) Assignee: VERONA PHARMA PLC, London (GB)

(21) Appl. No.: 14/342,490

(22) PCT Filed: Sep. 6, 2012
(86) PCT No.: PCT/GB2012/052190

§ 371 Date: Apr. 24, 2014

Related U.S. Application Data
(60) Provisional application No. 61/531,432, filed on Sep. 6, 2011.

(51) Int. Cl. A61K 31/167; A61K 9/00; A61K 9/14

(52) U.S. Cl. CPC A61K 31/167; A61K 9/14; A61K 9/0073; A61K 9/0075; A61K 9/0078
USPC 424/489; 514/616

(57) ABSTRACT

The invention is directed towards carcainium in the form of a salt having an anion An⁻, wherein An⁻ is an anion of pharmaceutically acceptable acid for use in the treatment and/or suppression of cough, tussive attacks or tussive episodes in a patient.

This application claims priority from U.S. Patent Application No. 61/531,432 filed 6 Sep. 2011, which is incorporated by reference.

### BACKGROUND OF THE INVENTION

Cough is the most common respiratory ailment for which patients seek medical help. It is a very common problem in medical practice as it accompanies a great variety of viral or bacterial infections including pneumonia, cold, flu and some underlying diseases, such as asthma, emphysema, lung cancer, etc.

Cough is a natural response to mechanical and chemical irritation of trachea and bronchi. The physiological role of cough is to prevent aspiration of foreign objects or excess secretion within the respiratory tract and to remove such objects or secretion or exudates from the trachea and bronchi.

Most of the current cough remedies are of limited effectiveness. Those that can be more effective are limited by their serious side effects. While there are several agents available on the market, most of these agents cause secondary undesirable symptoms, such as drowsiness, tiredness, gastrointestinal disturbances, and some of these agents, such as for example codeine, are also addictive.

Acute severe episodes of cough, although often limited in duration, can still be very troublesome. However, the condition of chronic cough (which persists in a troublesome form for more than eight weeks) is a serious debilitating condition estimated to affect some 14% of the population. It has an adverse effect on quality of life for many sufferers.

Over ten years ago, there was an initial suggestion that carcainium chloride might be useful in the treatment and/or prevention of cough. Thus, U.S. Pat. No. 6,362,197, filed in 1999, mentioned that carcainium chloride might have this activity. That conclusion was based on experiments carried out in an animal model in which cough was induced by citric acid aerosol. U.S. Pat. No. 6,362,197 did not contain any clinical data showing efficacy in treating cough in humans. Rather, the document simply assumed that clinical efficacy in humans would be achieved, based on the results from the animal model.

When carcainium chloride was first postulated as an anti-tussive, its structural similarity to known local anaesthetics was noted. It was assumed at the time that any anti-tussive activity was mediated by activity as a local anaesthetic. The expected side effects were thus local oropharyngeal numbing, impairment or loss of gag reflex and/or impairment or loss of the tracheal aspiration reflex.

Shortly after U.S. Pat. No. 6,362,197 was filed, it became apparent that the assumptions made in that document were not correct. Thus, a press release from Nortran Pharmaceuticals Inc. on 15 Dec. 2000 reported the results of a Phase II clinical trial on carcainium chloride in healthy human volunteers. This clinical trial was a blinded placebo-controlled cross-over trial, in which the primary endpoint was to determine whether carcainium chloride could increase the amount of irritant required to induce cough in the subjects. This clinical trial clearly established that, contrary to the assumptions made in U.S. Pat. No. 6,362,197, carcainium chloride had no statistically significant ability to inhibit cough. These clinical trial results rapidly became known in the art. Since 2000, it has been common general knowledge in this field that carcainium chloride does not have clinical efficacy in treating cough in humans.

### SUMMARY OF THE INVENTION

It is a finding of the present invention that, contrary to the widespread understanding since 2000, carcainium salts can in fact provide an effective therapy for cough in humans. That conclusion is based on a new clinical trial in which carcainium chloride is seen to have a statistically significant therapeutic effect in treatment and/or suppression of cough in patients suffering from interstitial lung disease.

It is a further finding of the present invention that the anti-tussive activity of carcainium salts is not mediated by local anaesthetic activity. Thus, the results in the clinical trial demonstrate that carcainium salts have efficacy as anti-tussives in human patients at dosages at which the salts have no local anaesthetic activity. This is highly significant. It means that carcainium salts can act as anti-tussive agents in patients without the local side effects which characterise the use of local anaesthetics. Such side effects include local oropharyngeal numbing, impairment or loss of gag reflex and/or impairment or loss of the tracheal aspiration reflex.

Accordingly, the present invention is directed towards carcainium in the form of a salt having an anion An⁻, wherein An⁻ is an anion of pharmaceutically acceptable acid, said carcainium salt being for use in the treatment and/or suppression of cough, tussive attacks or tussive episodes in a patient.

The invention further provides use of carcainium in the form of a salt having an anion An⁻, wherein An⁻ is an anion of pharmaceutically acceptable acid, in the manufacture of a medicament for the treatment and/or suppression of cough, tussive attacks or tussive episodes in a patient.

The invention further provides a method of treatment and/or suppression of cough, tussive attacks or tussive episodes in a patient, which method comprises administering to said patient a therapeutically effective amount of carcainium in the form of a salt having an anion An⁻, wherein An⁻ is an anion of a pharmaceutically acceptable acid.

The invention further provides an inhalable composition comprising carcainium in the form of a salt having an anion An⁻, wherein An⁻ is an anion of pharmaceutically acceptable acid, which composition is a dry powder aerosol or a nebulized aerosol, and wherein the particles present in said aerosol have a mass median aerodynamic diameter (MMAD) of from about 3 μm to about 10 μm.

The invention further provides an inhalable composition comprising carcainium in the form of a salt having an anion An⁻, wherein An⁻ is an anion of pharmaceutically acceptable acid, which composition is a dry powder aerosol or a nebulized aerosol, and wherein the particles present in said aerosol have a mass median aerodynamic diameter (MMAD) of from about 3 μm to about 10 μm, for use in the treatment and/or suppression of cough, tussive attacks or tussive episodes in a patient.

The invention further provides use of an inhalable composition comprising carcainium in the form of a salt having an anion An⁻, wherein An⁻ is an anion of pharmaceutically acceptable acid, which composition is a dry powder aerosol or a nebulized aerosol, and wherein the particles present in said aerosol have a mass median aerodynamic diameter (MMAD) of from about 3 μm to about 10 μm, in the manufacture of a medicament for the treatment and/or suppression of cough, tussive attacks or tussive episodes in a patient.

The invention further provides a method of treatment and/or suppression of cough, tussive attacks or tussive episodes in a patient, which method comprises administering to said patient a therapeutically effective amount of an inhalable composition comprising carcainium in the form of a salt having an anion An⁻, wherein An⁻ is an anion of pharmaceutically acceptable acid, which composition is a dry powder aerosol or a nebulized aerosol, and wherein the particles present in said aerosol have a mass median aerodynamic diameter (MMAD) of from about 3 μm to about 10 μm.

The invention further provides a dry powder inhaler or metered dose inhaler comprising a dry powder of carcainium in the form of a salt having an anion An⁻, wherein An⁻ is an anion of pharmaceutically acceptable acid, which inhaler delivers a dry powder aerosol of carcainium salt and wherein the particles present in said aerosol have a mass median aerodynamic diameter (MMAD) of from about 3 μm to about 10 μm.

The invention further provides an electronic nebulizer comprising a solution of carcainium in the form of a salt having an anion An⁻, wherein An⁻ is an anion of pharmaceutically acceptable acid, which nebulizer aerosolizes the solution of carcainium salt into an aerosol and wherein the particles present in said aerosol have a mass median aerodynamic diameter (MMAD) of from about 3 μm to about 10 μm.

### DETAILED DESCRIPTION OF THE INVENTION

Carcainium is the compound N,N-Bis-(phenylcarbamoylmethyl) dimethylammonium, and is used in the form of a salt having an anion An⁻, wherein An⁻ is an anion of a pharmaceutically acceptable acid. The carcainium salt thus has the following chemical structure.

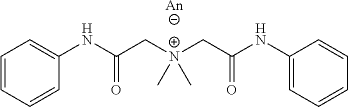

Typically, the pharmaceutically acceptable acid is hydrochloric, hydrobromic, benzenesulfonic (besylate), benzoic, camphorsulfonic, ethanesulfonic, fumaric, gluconic, glutamic, isethionic, maleic, malic, mandelic, methanesulfonic, mucic, nitric, pamoic, pantothenic, succinic, p-toluenesulfonic, phosphoric, sulphuric, citric, tartaric, lactic or acetic acid, with hydrochloric acid and hydrobromic acid preferred, and hydrochloric acid most preferred.

The carcainium salt is preferably the chloride salt, which is the compound N,N-Bis-(phenylcarbamoylmethyl) dimethylammonium chloride or carcainium chloride. Carcainium chloride has the following chemical structure.

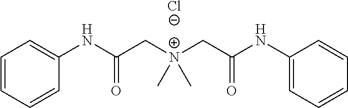 [SMILES: [Cl-] ; C[N+](C)(CC(=O)Nc1ccccc1)CC(=O)Nc1ccccc1]

Typically, the carcainium salt is delivered as a dry powder aerosol or a nebulized aerosol. The dry powder aerosol or nebulized aerosol preferably has particles which have a mass median aerodynamic diameter (MMAD) of from about 3 μm to about 10 μm, more preferably from about 4 μm to about 5.5 μm. As a skilled person will appreciate, when the carcainium salt is delivered as a nebulized aerosol, the reference to particle diameters defines the MMAD of the droplets of the aerosol. The mass median aerodynamic diameter (MMAD) can be measured by any suitable technique known to those skilled in the art, such as laser diffraction. Such particle sizes are preferred for effective delivery of the drug into the conducting and central airways.

Typically, the carcainium salt is formulated as a dry powder. Alternatively, it can be formulated as a solution, and then aerosolized and delivered to the patient by inhalation of the aerosol. Thus, the inhalable composition of the invention is preferably a carcainium salt solution or carcainium salt dry powder.

Typically, the carcainium salt solution is delivered using a nebulizer, preferably an electronic nebulizer. Alternatively, a jet nebulizer may be used. The nebulizer is able to aerosolize the carcainium salt solution into an aerosol comprising particles with an MMAD of from about 3 μm to about 10 μm, preferably from about 4 μm to about 5.5 μm. Preferably, the electronic nebulizer is a PARI™ eFlow electronic nebulizer, a DeVilbiss UltraNeb ultrasonic nebulizer, an Omron MicroAir NE-U22V electronic nebulizer or an Aerogen Aerodose electronic nebulizer. More preferably, the electronic nebulizer (such as the PARI™ eFlow electronic nebulizer or a DeVilbiss UltraNeb ultrasonic nebulizer) is modified to comprise a vibrating perforate membrane.

Typically, the carcainium salt solution comprises from about 10 to about 200 mg of carcainium salt dissolved in from about 1 to about 20 ml of a solvent. The solvent generally is normal or diluted saline. Normal saline means water solution containing 0.9% (w/v) NaCl. Diluted saline is normal saline diluted to from 1/20 to 9/10 normal strength.

Typically, the carcainium salt solution is packaged in a sealed low density polyethylene vial under sterile conditions for storage or in a two component packaging comprising a dry or lyophilized carcainium salt in one component and a normal or diluted saline in a second component.

Typically, the carcainium salt solution is specifically formulated for inhalation, and thus is preferably preservative free. The osmolality, pH, and viscosity are preferably optimized to be adequate for nebulization, for example via an electronic nebulizer.

Typically, the carcainium salt solution has an osmolality between 150 and 550 mOsm/kg, ion concentration between 31 and 300 mM of the permeant anion, pH between 5.5 and 7.0 and viscosity lower than 1.5 centipoise. The carcainium salt concentration is 5 to 80 mg per ml of saline, for example 10, 40, or 80 mg per ml of saline. Other than saline, there are preferably no other preservatives present which could cause secondary side effects.

Control of the pH of the carcainium salt solution is important for efficacious delivery of the nebulized drug. When the drug aerosol is either more acidic or basic than physiological pH, the patient may experience certain side effects, including bronchospasm. In particular, any aerosol with a pH below 4.5 or over 8.5 results is more likely to result in lung irritation accompanied by severe bronchospasm, exacerbated cough, and inflammatory reactions. A preferred pH is thus from 5.5 to 7.0.

Preferably the carcainium salt solution has an osmolality of 275 to 300 mOsm/kg and a pH of from 5.5 to 7.0. Preferably the carcainium salt solution has an osmolality of 275 and 300 mOsm/kg and a concentration of between 31 mM and 300 mM. More preferably the carcainium salt solution has an osmolality of 275 to 300 mOsm/kg, a pH of from 5.5 to 7.0 and a chloride concentration of between 31 mM and 300 mM.

Typically, the carcainium salt dry powder is delivered using a dry powder inhaler or metered dose inhaler.

Typically, the dry powder inhaler is a Clickhaler, Novolizer, Certihaler, Diskus, Multihaler, Gyrohaler (Vectura Group plc), Aerolizer, Handihaler or Tubospin (PH&T S.p.A.), Acu-Breathe unit (Respirics, Inc.), Conix (Cambridge Consultants Limited), Miat Monohaler (Cyclohaler), Eclipse (Sanofi-Aventis), e-flex (Microdrug AG), Flowcaps (Hovione), Prohaler (Valois Pharm), DirectHaler (Trimel BioPharma), Single Dose SDD (Manta technologies), Monodose (Miat SpA), TwinCaps (Hovione), GenX (CCL), SkyeHaler (SkyePharma), EasyHaler (Orion Pharma), or Taifun (Akela Pharma Inc.), with Clickhaler, Novolizer, Diskus and Aerolizer being the preferred dry powder inhalers.

Typically, the metered dose inhaler is an Airomir, Ventolin HFA, QVAR, Atrovent HFA or Clenil-HFA, with Airomir, Ventolin HFA and QVAR being the preferred metered dose inhalers.

Typically, the carcainium salt dry powder is prepared by milling, spray drying, fluidized spray drying, spray congealing, micronization, controlled crystallization, co-crystallization, ultrasound assisted crystallization, freeze drying or particle precipitation to the powder having a particle size with a mass median aerodynamic diameter from about 3.5 μm to about 10 μm, preferably from about 4 μm to about 5.5 μm. The dry powder composition may additionally comprise an excipient such as lactose, lysine or leucine.

Typically, the patient whose cough, tussive attacks or tussive episodes are treated and/or suppressed is human.

As discussed above, carcainium salts have efficacy as anti-tussives in human patients at dosages at which the salts have no local anaesthetic activity. Accordingly, the present invention also provides a said carcainium salt for use in the treatment and/or suppression of cough, tussive attacks or tussive episodes in a patient, wherein said carcainium salt acts by a mechanism independent of local anaesthesia.

The invention also provides a said carcainium salt for use in the treatment and/or suppression of cough, tussive attacks or tussive episodes in a patient, without causing any substantial local anaesthetic effect. Local anaesthetic activity in inhaled medicaments causes side effects such as oropharyngeal numbing, impairment or loss of gag reflex and/or impairment or loss of the tracheal aspiration reflex. Typically, therefore, said carcainium salt is for use in the treatment and/or suppression of cough, tussive attacks or tussive episodes in a patient, without causing any substantial oropharyngeal numbing, impairment or loss of gag reflex and/or impairment or loss of the tracheal aspiration reflex.

Typically, said carcainium salt is for use in the treatment and/or suppression of cough, tussive attacks or tussive episodes in a patient suffering from or susceptible to bronchospasm, oropharyngeal numbing, impairment or loss of gag reflex and/or impairment or loss of the tracheal aspiration reflex, and more typically in a patient suffering from or susceptible to oropharyngeal numbing, impairment or loss of gag reflex and/or impairment or loss of the tracheal aspiration reflex. The carcainium salt is particularly effective in such patients, and also due to the low systemic side effects associated with the invention.

Typically, said carcainium salt is for use in the treatment and/or suppression of cough, tussive attacks or tussive episodes in a patient, wherein said salt is (a) for use during a surgical or invasive procedure, or (b) for chronic use. A preferred surgical or invasive procedure where said carcainium salt can be used is bronchoscopy. Chronic use typically means administration of said carcainium salt twice a day or more, for example up to five times per day, or administration of said carcainium salt once a day or more over a period of one week or more, for example over a period of two weeks or more.

Typically, the carcainium salt is administered such that systemic exposure of carcainium salt following delivery to the patient as measured by peak plasma concentration is less than 800 ng/ml, more preferably less than 500 ng/ml, more preferably less than 100 ng/ml, and most preferably less than 70 ng/ml. The plasma concentration of carcainium salt can be measured by any suitable technique known to those skilled in the art, such as a liquid chromatography/tandem mass spectrometry (LC/MS/MS) assay method. One such suitable method is described in the Examples below.

The origin of the cough to be treated by the present invention is not particularly limited, and can include virtually any respiratory disorder, such as chronic obstructive pulmonary disease, asthma, tuberculosis, bronchitis, bronchiectasis, suppurative pulmonary disease, respiratory malignancies, allergy, cystic fibrosis, pulmonary fibrosis, respiratory tract inflammation, emphysema, pneumonia, lung cancer, lung neoplasia, sore throat, common cold, influenza, respiratory tract infection, bronchoconstriction, sarcoidosis, smoker's cough, chronic non-productive cough, neoplastic cough; cough due to gastroesophageal reflux, inhalation of irritants, smoke, smog, dust, presence of foreign bodies, air pollution or angiotension converting enzyme (ACE) inhibitor therapy, or acute or chronic cough resulting from or connected with a viral or bacterial infection of the upper airways; or intractable cough resulting from or connected with another underlying disease.

Typically, the underlying disease may be chronic obstructive pulmonary disease, asthma, tuberculosis, bronchitis, bronchiectasis, suppurative pulmonary disease, respiratory malignancies, allergy, cystic fibrosis, pulmonary fibrosis, respiratory tract inflammation, emphysema, pneumonia, lung cancer, lung neoplasia, soar throat, common cold, influenza, respiratory tract infection, bronchoconstriction, sarcoidosis, gastroesophageal reflux, smoker's cough, chronic non-productive cough, neoplastic cough, or acute or chronic cough resulting from or connected with a viral or bacterial infection of the upper airways.

Alternatively, the origin of the cough to be treated by the present invention may be interstitial lung disease. In that instance, the cough, tussive attacks or tussive episodes result from interstitial lung disease. Interstitial lung diseases affect the interstitium, which is the tissue and space around the air sacs of the lungs, and in particular the alveolar epithelium, pulmonary capillary endothelium, basement membrane, perivascular and perilymphatic tissues.

Interstitial lung disease may be irritant-induced (for example by silica dust or asbestos) or drug induced (for example by antibiotics, chemotherapeutic drugs, antiarrhythmic agents, or statins). Interstitial lung disease may also arise from connective tissue diseases (such as systemic sclerosis, polymyositis, dermatomyositis, systemic lupus erythematosus or rheumatoid arthritis), from infection (such as atypical pneumonia, pneumocystis pneumonia (PCP), tuberculosis, chlamydia trachomatis or respiratory syncytial virus) or from malignancy (such a lymphangitic carcinomatosis). Interstitial lung disease may also be idiopathic, arising from for example sarcoidosis, idiopathic pulmonary fibrosis, Hamman-Rich syndrome or Antisynthetase Syndrome.

Typically, the carcainium salt is administered such that substantially whole dose of the drug is delivered to specific target areas, namely the trachea, carina and bronchi, while minimizing the deposition of the drug in other areas where it could cause undesirable local side effects or more easily enter the systemic circulation and cause undesirable side effects.

As discussed above, the dry powder aerosol or nebulized aerosol which has particles having a mass median aerodynamic diameter (MMAD) of from about 3 μm to about 10 μm is preferred for effective delivery of the drug into the conducting and central airways. The carcainium salt is thus typically targeted to the conducting and central airways of the patient. The central airways are the region of the respiratory tract defined by trachea, carina and bronchi. The carina means the ridge separating the opening the right and left main bronchi at their junction with the trachea. Accordingly, the carcainium salt is typically delivered to the patient such that it does not cause bronchospasm, oropharyngeal numbing, impairment or loss of gag reflex, impairment or loss of the tracheal aspiration reflex or systemic exposure that leads to adverse side effects.

Efficacy of administration of carcainium salt is measured by the amount of the drug needed for cough abatement, by the frequency of administration needed to suppress tussive attacks or episodes, by the time necessary for delivery of the drug amount and by the percentage of the drug deposited in the specific target areas, namely in trachea, carina and bronchi as well as a lack of deposition in the other areas.

The magnitude of the therapeutic or prophylactic dose of carcainium salt required for the treatment and/or suppression of cough, tussive attacks or tussive episodes in a patient will depend upon the severity and nature of the condition being treated and the route of administration. The dose and the frequency of the dosing will also vary according to age, body weight and response of the individual patient. Typically, the daily dose is determined based on the weight of the patient. Preferably, the daily dose is 0.5 to 5 mg/kg, for example about 1.0 mg/kg, based on the weight of the patient.

Typically, the total daily dose of carcainium salt is from about 5 mg to about 300 mg. This may be delivered in a single dose or in repeated doses, for example up to five times a day, but is preferably delivered as a single dose. By daily dose it is meant the total quantity of compound of the invention administered to the patient in a day.

Typically the daily dose is a single metered nominal dose of from about 5 mg to about 300 mg. A metered nominal dose refers to the quantity of drug substance contained in the metering chamber of the delivery device and is normally expressed as quantity per actuation.

Upon actuation, the drug substance leaves the device and becomes available to the patient as a “delivered dose”. The delivered dose is normally smaller than the metered nominal dose, due to the mechanics of the device. Thus, the delivered dose is the amount of the drug which is available at the mouth for inhalation. The delivered dose can be measured using standard techniques known to those skilled in the art. Typically, the delivered dose is from about 4.5 mg to about 275 mg.

Thus, the invention also provides a dry powder inhaler or metered dose inhaler comprising a dry powder of carcainium salt, which inhaler delivers a dry powder aerosol of carcainium salt and wherein the particles present in said aerosol have a mass median aerodynamic diameter (MMAD) of from about 3 μm to about 10 μm, and which inhaler is configured to deliver (a) a metered nominal dose of about 5 mg to about 300 mg carcainium salt, and/or (b) a delivered dose of about 4.5 mg to about 275 mg carcainium salt.

The invention also provides an electronic nebulizer comprising a solution of carcainium salt, which nebulizer aerosolizes the solution of carcainium salt into an aerosol and wherein the particles present in said aerosol have a mass median aerodynamic diameter (MMAD) of from about 3 μm to about 10 μm, and which nebulizer is configured to deliver (a) a metered nominal dose of about 5 mg to about 300 mg carcainium salt, and/or (b) a delivered dose of about 4.5 mg to about 275 mg carcainium salt.

Upon improvement of a patient's condition, a maintenance dose of carcainium salt may be administered, if necessary. Subsequently, the dosage or frequency of administration, or both, may be reduced, as a function of the symptoms, to a level at which the improved condition is retained. When the symptoms have been alleviated to the desired level, treatment should cease. The patient may, however, require intermittent treatment on a long-term basis upon any recurrence of disease symptoms.

It will be understood, however, that the total daily usage of the carcainium salt will be decided by the attending physician within the scope of sound medical judgment. The specific dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Any suitable route of administration may be employed to provide an effective dosage of the compounds of the present invention, although administration by inhalation is preferred, most preferably in aerosol form. Suitable forms of administration include, but are not limited to, inhalation (delivered by, e.g., metered dose inhaler, jet nebulizer, ultrasonic nebulizer, dry powder inhaler, etc.), nasal sprays, nebulization, oral administration such as via tablets, capsules, lozenges, syrups, sprays, suspensions, elixirs, gargles, and other liquid preparations, aerosol foams, parental administration, and sublingual administration. Topical administration to the lung via inhalation is particularly preferred.

The compositions of the present invention can include pharmaceutically acceptable carriers and other conventional additives, including aqueous based carriers, co-solvents such as ethyl alcohol, propylene glycol and glycerin, fillers, lubricants, wetting agents, flavoring agents, coloring agents, emulsifying, suspending or dispersing agents, suspending agents, etc. For aerosol delivery of the compounds of the present invention, pharmaceutically acceptable diluents, carriers, and/or propellants may be included in the compositions for use in appropriate devices. These are prepared by procedures well known to those skilled in the art (see e.g., Medication Teaching Manual, 5th Ed., Bethesda, Md., American Society of Hospital Pharmacists, 1991).

The compositions of the present invention may optionally include other known therapeutic agents, including decongestants such as pseudoephedrine HCl, phenylephrine HCl and ephedrine HCl, non-steroidal anti-inflammatory drugs such as acetaminophen, aspirin, phenacetin, ibuprofen and ketoprofen, expectorants such as glyceryl guaiacolate, terpin hydrate and ammonium chloride, antihistamines such as chlorpheniramine maleate, doxylamine succinate, brompheniramine maleate and diphenhydramine hydrochloride.

The carcainium salt can be administered in combination with (a) one or more additional anti-tussive agents and/or (b) one or more bronchodilators. Preferred additional anti-tussive agents are menthol or codeine. A preferred bronchodilator is N-{2-[(2E)-2-(mesitylimino)-9,10-dimethoxy-4-oxo-6,7-dihydro-2H-pyrimido[6,1-a]-isoquinolin-3(4H)-yl]ethyl}urea (which is known by the code RPL-554).

Accordingly, the present invention also provides a combination comprising a carcainium salt, and (a) one or more additional anti-tussive agents and/or (b) one or more bronchodilators. The combination is preferably for use in the treatment and/or suppression of cough, tussive attacks or tussive episodes in a patient.

The invention further provides a carcainium salt for use in the treatment and/or suppression of cough, tussive attacks or tussive episodes in a patient, by co-administration with (a) one or more additional anti-tussive agents, and/or (b) one or more bronchodilators. Co-administration can be simultaneous, concurrent, separate or sequential.

The invention further provides (a) one or more additional anti-tussive agents and/or (b) one or more bronchodilators, for use in the treatment and/or suppression of cough, tussive attacks or tussive episodes in a patient, by co-administration with a carcainium salt. Co-administration can be simultaneous, concurrent, separate or sequential.

The present invention further provides a product comprising a carcainium salt and (a) one or more additional anti-tussive agents and/or (b) one or more bronchodilators, as a combined preparation for simultaneous, concurrent, separate or sequential use in the treatment and/or suppression of cough, tussive attacks or tussive episodes in a patient.

Carcainium salts such as carcainium chloride can be synthesized as described in U.S. Pat. No. 6,362,197 and Belgian Patent No. 614,154, which follows from Swedish Patent 1779/61, the disclosures of which are herein incorporated by reference. A conventional route of synthesis involves three steps and can be described (as in the aforementioned patent; see also T. Takahashi, J. Okada, M. Hori, A. Kato, K. Kanematsu, and Y. Yamamoto, J. Pharm. Soc. Japan 76, 1180-6 (1956)) as follows:

### i) Chloroacetanilide

To a chilled solution of aniline (37.2 g, 0.40 mol) and potassium carbonate (66.4 g, 0.48 mol) in chloroform (200 ml) was added dropwise via cannula a solution of chloroacetylchloride (49.6 g, 0.44 mol) in chloroform (100 ml) and the reaction mixture was heated to 55° C. for 90 min. To the cooled reaction mixture was then added water (300 ml), the organic layer was collected and the aqueous layer was extracted twice more with chloroform (2×100 ml). The combined organic layers were dried over sodium sulfate and evaporation of the solvent in vacuo provided the crude product. The product was purified via extraction through a Soxhlet apparatus with diethyl ether to provide 22 g of the desired chloroacetanilide. m.p. 133-135° C.

### ii) Dimethylaminoacetanilide

A mixture of chloroacetanilide (10.0 g, 59 mmol) in dimethylamine, 40% wt in water (100 ml) was refluxed for 4 hours. The cooled reaction mixture was partitioned between dichloromethane (100 ml) and 1M NaOH aqueous solution (100 ml). The aqueous layer was extracted twice more with dichloromethane (2×100 ml), the combined organic layers were concentrated in vacuo to a volume of approximately 100 ml and washed with water (2×100 ml) in order to remove the remaining dimethylamine The organic layer was collected, dried over sodium sulfate and the solvent evaporated in vacuo to provide 10.2 g (97% yield) of the pure dimethylaminoacetanilide.

N,N-Bis-(phenylcarbamoylmethyl)dimethylammonium chloride (carcainium chloride)

A mixture of chloroacetanilide (10.1 g, 59.5 mmol), dimethylaminoacetanilide (10.7 g, 60 mmol) and potassium iodide, 99+% (0.1 g, 0 6 mmol) in dry xylene (30 ml) was refluxed for 1 hour and then allowed to stand overnight to ambient temperature. The solvent was decanted and the remaining gummy solid was triturated in diethyl ether in order to obtain a whitish powder. The resulting solid was collected and recrystallized in a mixture of ethanol and diethyl ether to provide 9.3 g (45% yield) of the desired ammonium salt. m.p. 177-178° C.

The following Examples illustrate the invention.

### EXAMPLES

### Example 1

### A Clinical Study in Humans

The aim of the clinical study was to determine the clinical effectiveness and safety of carcainium chloride by the inhaled route in hospital in-patients with intractable, persistent cough due to interstitial lung disease.

A double blind, randomized, placebo-controlled, cross-over study design was used to assess the effect of carcainium chloride as an anti-tussive in patients with interstitial lung disease.

The study was constructed as an adaptive contingency trial. In such a trial the outcome of each “test” of the drug against placebo is scored as either a positive or negative result (i.e. a binary decision, rather than a quantitative measure). Based on the outcome of this “test”, the trial either continues or halts. Patients attended two study visits where they were randomised to receive either:
carcainium chloride at a dose of 1.0 mg/kg on the first study visit followed by placebo (sodium chloride 0.9%) on the second visit; or
placebo (sodium chloride 0.9%) on the first visit followed by carcainium chloride at 1.0 mg/kg on the second visit.

Assessment of each patient was based on the following criteria.
1 A physician's professional judgment of individual patient responses in a double blind crossover study with carcainium chloride and placebo in terms of anti-tussive action.
2. Each patient's subjective comfort using a visual analogue scale (VAS), pre- and post-treatment.
3. Each patient's coughs recorded in pre- and post-treatment periods for active and placebo treatments.

### Preparation and Administration of Active and Placebo Samples

Carcainium chloride is a fine white dry powder and was provided in tightly closed vessels and stored in dark at room temperature upon receipt. The vehicle used for the dilution of carcainium chloride and for the placebo was 0.9% NaCl injection.

Patients were then administered the active or placebo sample, according to the above schedule, as an aerosol generated using an ultrasonic nebulizer (DeVilbiss Ultraneb).

### 1. Physician Assessment

In 8 out of 8 trials, the two physicians acting in concert successfully identified the active treatment carcainium chloride. The outcome of this assessment or “test” after each study treatment administered was either a success in which an ‘anti-tussive’ effect was observed (i.e. positive) or a failure which was defined as no change from baseline or pro-tussive effect (i.e. negative). The clinical investigator decided on a positive or negative outcome for each “test” in a blinded fashion. The statistical significance of this finding is <0.05 by contingency table, 0.05-0.01 by Chi square.

### 2. Patient Comfort Score

This was assessed on a 0-10 equal interval scale in the pre-drug and post-drug periods for active drug and placebo periods. A statistically significant improvement in patient well-being was observed with patients treated with carcainium chloride as compared to patients treated with placebo (p=0.0140 when assessed by sum of signed ranks).

### 3. Number of Coughs Recorded Electronically

The frequency of cough (number per unit time) was recorded using a semi-automated system which records cough epochs (sounds) that are counted by a qualified technician. There was a statistically significant treatment effect (p=0.0007) associated with carcainium chloride as compared to placebo.

### Summary of the Results from the Clinical Study

Preliminary analysis of the clinical study results showed that carcainium chloride has marked anti-tussive activity which could be detected with subjective measures by two physicians when tested in a double blind randomized cross over contingency trial. All three measures of carcainium chloride's effectiveness revealed a statistically significant anti-tussive response. The patient comfort score (VAS) showed marked improvement in patient well-being as did the more objective measure involving the number of coughs recorded electronically.

### Example 2

### Measurement of Particle Size Distribution

Carcainium chloride aerosols corresponding to those used in the above clinical study were generated from the same drug product batch and using the same ultrasonic nebulizer (DeVilbiss Ultraneb). The particle size distributions of these carcainium chloride aerosols were measured and analyzed using a Malvern Spraytec with inhalation cell attachment system. Results from two replicate experiments showed an average value of about 5.38 μm for the Spraytec volume median diameter [Dv(50)].

It is generally accepted in the literature that laser diffraction techniques will agree with aerodynamic techniques when measuring spherical particles of unit or similar density, for example water or aqueous solution aerosols such as those generated above. Therefore the above average Dv(50) value of about 5.38 μm can be taken to correspond to an average mass median aerodynamic diameter (MMAD) value of about 5.38 μm for the particle size distribution of the carcainium chloride aerosol used in the above clinical study.

### Example 3

### Measurement of Carcainium Chloride Concentrations in Human Plasma

A liquid chromatography/tandem mass spectrometry (LC/MS/MS) assay method was developed, qualified, and implemented for the quantitation of carcainium chloride levels in K₂EDTA human plasma samples collected from the human clinical study detailed above.

Initially, carcainium chloride was dissolved in deionized water to provide an initial standard or quality control (QC) stock solution at a concentration of 1000 μg/mL. Serial dilutions were carried out with deionized water to provide secondary stock solutions for subsequent preparation of plasma calibration standards or QC samples according to Table 1 below. The internal standard lidocaine was dissolved in deionized water to provide an initial stock solution at a concentration of 1000 μg/mL. The initial internal standard stock solution was then serially diluted with deionized water to provide a spiking stock solution concentration of 50 ng/mL.

TABLE 1

| Calibration | | | | |
|---|---|---|---|---|
| Standards or | Stock | | Vol of Human | Final |
| QC Samples | Solutions | Vol of Stock | K₂EDTA | Conc. |
| (ng/mL) | Used (ng/mL) | Solution (μL) | Plasma (μL) | (ng/mL) |
| STD-500 | STD-5000 | 10 | 100 | 500 |
| STD-400 | STD-4000 | 10 | 100 | 400 |
| STD-100 | STD-1000 | 10 | 100 | 100 |
| STD-20 | STD-200 | 10 | 100 | 20 |
| STD-10 | STD-100 | 10 | 100 | 10 |
| STD-2.0 | STD-20 | 10 | 100 | 2.0 |
| STD-1.0 | STD-10 | 10 | 100 | 1.0 |
| STD-0.5 | STD-5 | 10 | 100 | 0.5 |
| QC-400 | QC-4000 | 10 | 100 | 400 |
| QC-80 | QC-800 | 10 | 100 | 80 |
| QC-1.5 | QC-15 | 10 | 100 | 1.5 |

A 10 μL aliquot of calibration standard stock solution or QC stock solution was transferred into individual 16×100 mm screw cap glass test tubes. For blank and blank with internal standard samples, 10 μL of deionized water was transferred instead. Human blank K₂EDTA plasma (100 μL) was then added to each tube. A 50 μL aliquot of of lidocaine internal standard spiking stock solution was transferred to each tube except for blank samples. A 50 μL aliquot of deionized water was added to blank samples. Samples were then vortex mixed.

A 100 μL aliquot of 1 M sodium hydroxide (NaOH) in deionized water was then added to each tube, followed by vortex-mixing. Methyl tert-butyl ether (3 mL) was then added to each tube followed by vortex-mixing for at least 20 seconds. The samples were then frozen at −80° C. for at least 10 minutes. The top layer was transferred to 13×100 mm glass test tubes.

The supernatant was dried under a gentle stream of nitrogen to complete dryness using a Turbovap or under a gentle stream of air using an air dryer. Each sample was reconstituted with 100 μL of a 1:4 (v/v) mixture of 0.1% FA in MeOH:0.1% FA in deionized water. To facilitate reconstitution, the mixture was vortex-mixed for 1 minute followed by sonication for 5 minutes before being transferred to a 250 μL vial and capped. All vials were centrifuged at 5,000 rpm for 5 min and an aliquot of 25 μL was injected for LC/MS/MS analysis.

For human plasma samples 100 μL of sample was transferred into individual 16×100 mm screw cap glass test tubes. A 10 μL aliquot of deionized water was then transferred to each tube. A 50 μL aliquot of lidocaine internal standard solution was transferred to each tube and vortexed to mix. Test samples were then processed the same as calibration standards and quality control samples mentioned above.

The calibration curves established above were used to determine the concentration of carcainium chloride in plasma samples from patients who had been administered 1 mg/kg carcainium chloride. This was found to be in the range of 1-50 ng/ml. This a measure of systemic exposure to the drug, and the low levels of systemic exposure of the drug observed are consistent with the general observation that no significant drug related adverse side effects were encountered in the human clinical study.

1. A method of treating and/or suppressing cough, tussive attacks or tussive episodes in a human subject suffering from interstitial lung disease, which method comprises the administration to the said subject of an effective amount of carcainium in the form of a salt having an anion An⁻, wherein An⁻ is an anion of a pharmaceutically acceptable acid.

2. The method of to claim 1, wherein the carcainium salt is carcainium chloride.

3-4. (canceled)

5. The method of claim 1, wherein the subject is suffering from or susceptible to oropharyngeal numbing, impairment or loss of gag reflex and/or impairment or loss of the tracheal aspiration reflex.

6. The method of claim 1, wherein (a) said carcainium salt is for use during a surgical or invasive procedure, or (b) said carcainium salt is for chronic use.

7. The method of claim 6, wherein said surgical or invasive procedure is bronchoscopy.

8. The method of claim 6, wherein said chronic use is administration of said carcainium salt twice a day or more, or administration of said carcainium salt once a day or more over a period of one week or more.

9. The method of claim 1, wherein the carcainium salt is formulated for delivery as an inhalable solution, suspension or dry powder such that systemic exposure of carcainium salt following delivery to the patient as measured by peak plasma concentration is less than 800 ng/ml.

10. The method of claim 8, wherein the daily dose of the carcainium salt is from about 5 mg to about 300 mg.

11. The method of claim 1, wherein the carcainium salt is administered to the conducting and central airways which are comprised of trachea, carina and bronchi.

12. The method of claim 1, wherein the patient is not concurrently administered local anaesthetics, steroids and/or bronchodilators.

13-16. (canceled)

17. The method of claim 1, wherein the carcainium salt is delivered as a dry powder aerosol or a nebulized aerosol and wherein the particles present in said aerosol have a mass median aerodynamic diameter (MMAD) of from about 3 μm to about 10 μm.

18. The method of claim 17, wherein the mass median aerodynamic diameter (MMAD) is from about 4 μm to about 5.5 μm.

19. The method of claim 17, wherein the carcainium salt is delivered as a nebulized aerosol.

20. The method of claim 19, wherein the nebulized aerosol is generated from a solution which comprises from about 10 to about 200 mg of carcainium salt dissolved in from about 1 to about 20 ml of a solvent.

21. The method of claim 20, wherein the solution is substantially free of preservatives.

22. The method of claim 17, wherein said nebulized aerosol is generated with a nebulizer.

23. The method of claim 1, wherein the carcainium salt is formulated as a dry powder aerosol.

24. The method of claim 23, wherein the dry powder aerosol is delivered by a dry powder inhaler.

25. The method of claim 23, wherein the dry powder aerosol is delivered by a metered dose inhaler.

26-27. (canceled)

28. The method of claim 22, wherein said nebulizer is an electronic nebulizer or jet nebulizer.
